(19) United States
(12) Patent Application Publication
(10) Pub. No.: US 2022/0115147 A1
(43) Pub. Date: Apr. 14, 2022

(54) CARE PATHWAY AND PHYSICIAN VISIT PATHWAY MANAGEMENT

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/500,899

(22) Filed: Oct. 13, 2021

Related U.S. Application Data
(60) Provisional application No. 63/204,622, filed on Oct. 14, 2020.

(51) Int. Cl. G16H 80/00; G06Q 10/06; G16H 20/00; G16H 10/60

(52) U.S. Cl. CPC G16H 80/00; G16H 10/60; G16H 20/00; G06Q 10/06312

(57) ABSTRACT

In an assisted living platform, users are provided with a dashboard (portal) for capturing their personal details, preferences and service needs, and service providers can provide details about themselves and the services they are able to provide.

### FIELD OF THE INVENTION

The invention relates to health management, scheduling of medical practitioner visits, and management of a care plan.

### BACKGROUND OF THE INVENTION

During a typical visit to a doctor, the patient may subsequently be presented with a care plan that defines follow-up tasks to be performed by the patient, some of which may require assistance by a medical practitioner, the taking of certain prescriptions, wound treatment, etc. They may also be asked to schedule a follow-up appointment with the doctor.

All of these activities require coordination among multiple people, such as the patient and a medical practitioner. e.g., a physio therapist. Often schedules need to be shifted and new appointments made. Patients forget and miss appointments. Patients forget to comply with a care plan, e.g., by failing to take their medication. Since many follow-up procedures generate additional costs, the issue of reimbursement and payer coverage has to be considered.

The patient's personal needs may conflict with those of an insurance provider (payer). For instance, a patient may wish to see a specific medical practitioner who is not covered by the patient's insurance plan, or the medical practitioner who is covered may not have an opening within a period of time that is acceptable to the patient, or the meeting with the medical practitioner may require an excessively long commute by the patient.

The present invention seeks to address these concerns in a streamlined manner.

### SUMMARY OF THE INVENTION

One aspect of the invention includes the generation of visit pathways (follow-ups with a physician) and management of tasks and the coordination of service providers that make up a care plan. This may involve an information capture and decision-making system (also referred to herein as an Optimizer), to identify potential service providers based on availability, proximity to the patient, insurance coverage, and customer rating profile. It may also take into account the ability of a service provider to perform multiple required tasks forming part of a care plan. It may choose an optimal person or present the patient with several options based on one or more of: insurance coverage, availability, proximity, and rating.

If a service provider becomes unavailable—the system may re-allocate a service provider in real time.

Another aspect of the invention is to keep a record of tasks performed and to monitor compliance. This may involve the capture of information using sensors to validate what the service provider is doing. These may include medical sensors and a service provider user device (SPUD) that allows a medical practitioner or other service provider to record the tasks performed. The data from the sensors and service provider user device may be used by the physician to update the information in the patient's electronic medical record (EMR). The SPUD may include a user interface defined, for example by a web application (web app) accessible through a browser (using Wifi or a cell phone connection) or a native mobile application (App) that is downloaded to the service provider's user device. The SPUD may also serve as a scheduling and management device to schedule the next appointment and define what task(s) will be performed at the next appointment.

Similarly, the patient may have a patient user device (PUD) with a user interface giving the patient an up-to-date overview of the tasks associated with a particular care plan and which tasks have been performed, as well as when, where, and through whom the next task(s) will be performed. It also allows the user to select or change service providers. The system may identify multiple service providers and present these to the patient to select one based on one or more selection criteria, including: out-of-pocket cost to the patient (related to insurance coverage), availability (timespan before service provider can see the patient), proximity (if patient has to travel to the service provider's location), gender of the service provider, and rating (based on one or more of feedback from other patients, experience level in general, and experience level with the particular task to be performed).

By including all tasks (services) and service providers under one system, it allows a patient to choose their optimum service provider (physician, clinician, hospital) based on insurance coverage, location, availability, rating, and other preferences.

Thus, according to the invention, there is provided a system for implementing a care plan for a patient, comprising a processor, memory in communications with the processor and configured with machine-readable code to define an algorithm, a data memory for storing data, at least one patient user device (PUD) with patient user interface, in communication with the processor, at least one service provider device (SPUD) with service provider user interface, in communication with the processor, and at least one medical provider user device (MUD) with patient user interface, in communication with the processor.

The data memory typically forms part of an information capture system, and the algorithm may define a decision-making system (also referred to herein as an Optimizer), to identify potential service providers based on parameters that include one or more of: availability, proximity to the patient, insurance coverage of the patient, customer rating profile, and gender of the service provider.

The Optimizer may also take into account the ability of a service provider to perform multiple required tasks forming part of a care plan.

The Optimizer may present the patient on the patient user interface with several options of service providers based on one or more of: insurance coverage of the patient, availability, proximity, and rating, or may identify an optimal service provider based on a ranking of said parameters.

The algorithm may include logic to verify compliance by the service provider in completing the one or more tasks allocated to the service provider, wherein compliance by the service provider may include data uploaded by the service provider via the user interface on the service provider's SPUD, including one or more of: time and details of the task, and digital input captured by the service provider using one or more medical sensors.

The Optimizer may re-allocate a service provider in real time if a service provider selected by a patient becomes unavailable or fails to verify compliance.

The algorithm may include logic to keep a record of tasks performed for a patient as part of the patient's care plan.

The service provider user interface preferably defines a scheduling and management system to schedule the next appointment and define what task(s) are to be performed and where they are to be performed.

The patient user interface may be arranged to provide the patient with an up-to-date overview of the tasks associated with a particular care plan and which tasks have been performed, as well as when, where, and through whom the next task(s) will be performed.

The patient user interface may also be arranged to allow the patient to select and change service providers.

Further, according to the invention, there is provided a system for implementing a care plan for a patient, comprising: a physician portal configured to receive care plan information, a service provider portal for entering service provider details and receive service requests, a patient portal for selecting one or more service providers based on a care plan for the patient, a processor, a data memory, and a control memory connected to the processor, wherein the control memory includes machine-readable code defining an algorithm for identifying suitable service providers for one or more tasks in the care plan and presenting them for selection to the patient via the patient portal. For purposes of this application the user interfaces will also be referred to interchangeably herein as portals or dashboards.

The portals may be defined by a web application or native local app.

The algorithm may take into account parameters that include one or more of: availability, proximity to the patient, insurance coverage for the patient, customer rating profile, and gender of the service providers.

Still further, according to the invention, there is provided a method of implementing a care plan for a patient, comprising: defining the tasks involved in the care plan, identifying suitable service providers for each task based on one or more of: availability, proximity to the patient, insurance coverage for the patient, customer rating profile, and gender of the service provider, the method further comprising allowing the patient to select a service provider for each task, and tracking compliance by one or more of: the patient and the selected service providers.

The defining of tasks in the care plan, the selection of service providers by the patient, and interaction with service providers may be provided via an Internet server system that defines a user portal and a service provider portal. The Internet server system may further define a physician portal for capturing the tasks involved in the care plan.

### BRIEF DESCRIPTION OF THE DRAWINGS

FIG. 1 shows one embodiment of a system of the invention;

FIG. 3 shows one embodiment of a user interface for a patient's user device;

FIG. 4 shows one embodiment of a user interface for a service provider's user device, and

FIG. 5 shows one embodiment of a flow chart depicting the logic defined by the algorithm controlling a processor of the system of the invention.

### DETAILED DESCRIPTION OF THE INVENTION

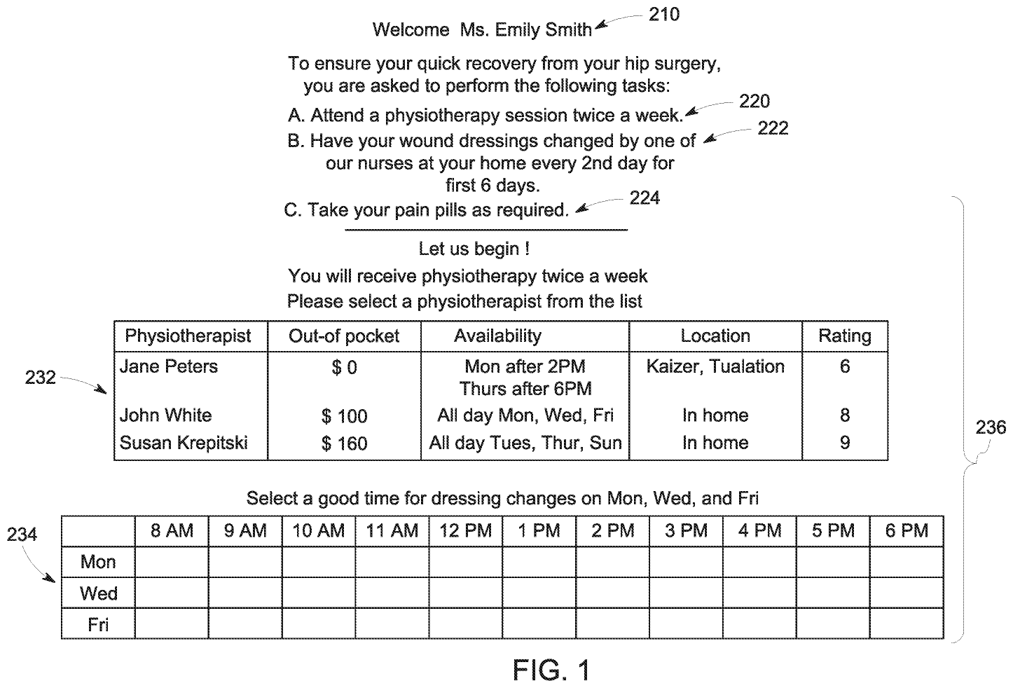

One embodiment of a system of the invention is shown in FIG. 1. The system 100 includes a processor with memory, and data storage, which in this embodiment comprises a server 102 and database 104. The server may be implemented as a centralized server, as a cloud server system such as Amazon Web Services (AWS) or as an edge computing system that performs some of the data processing and logic (which are discussed in more detail below) at a location closer to where the data is captured.

In this embodiment, the server 102 is connected by Wifi to multiple client devices, which comprise three groups of devices: Patient User Devices (PUDs) 110, Service Provider User Devices (SPUDs) 120, Medical Professional User Devices (MUDs) 130.

Each of these client devices (also referred to herein as user devices) supports a user interface (also referred to herein as a portal). For example, the user interface may be implemented using a native mobile application (App) that is downloaded to the user device, or may be based on accessing a web page hosted on the server 102 (or another server), i.e., a web application (web app) accessible through a browser (using Wifi or a cell phone connection).

The PUDs 110 are depicted in FIG. 1 as being implemented as smart phones, however they can comprise any devices that will support a user interface as discussed herein.

One embodiment of a PUD user interface is shown in FIG. 2, which allows patients to view a doctor's care plan for them and make certain selections. The user interface 200 includes a welcome message 210, and lays out a care plan for one or more discharge after-care programs. For instance, in this example, Ms. Emily Smith had a hip replacement and requires physiotherapy twice a week and dressing changes during the first week with pain medicine as needed. These are laid out as tasks 220, 222, 224.

Any actions or options available to the patient are then addressed in an Action section 230.

In this example the patient can select a physiotherapist from the table 232, based on their preferences, which in this case includes different out-of-pocket costs (based on insurance coverage), different availability of the physiotherapist, location of the session (at patient's home or at physiotherapists location), and a score from 1-10 (based on patient feedback, and experience level). In this embodiment, the gender of the service provider is inherent in the name of the provider. In another embodiment this information may be specifically mentioned or form part of the metadata allowing a search engine to be integrated into the PUD to allow the user to add certain filters to the service provider options, e.g., specifying that only female, in-plan providers be shown.

Once the patient makes a physiotherapist selection a confirmation message confirms the selection (not shown) and the patient is presented with a calendar of the selected physiotherapist defining the open times available for scheduling an appointment.

The patient in this example is then provide with a time-selection option 234 to have a nurse come to the patient's home to change the bandages/dressings (which in this case is Monday, Wednesday and Friday for the week following the surgery).

Once the patient has made this selection, the user interface 200 brings up a calendar of all tasks that will be performed or required of the patient as part of the care plan (not shown).

In this example the calendar will highlight Mondays, Wednesdays, and Fridays with the selected times for the dressing change. It will also populate the calendar with the physiotherapy sessions, times and locations.

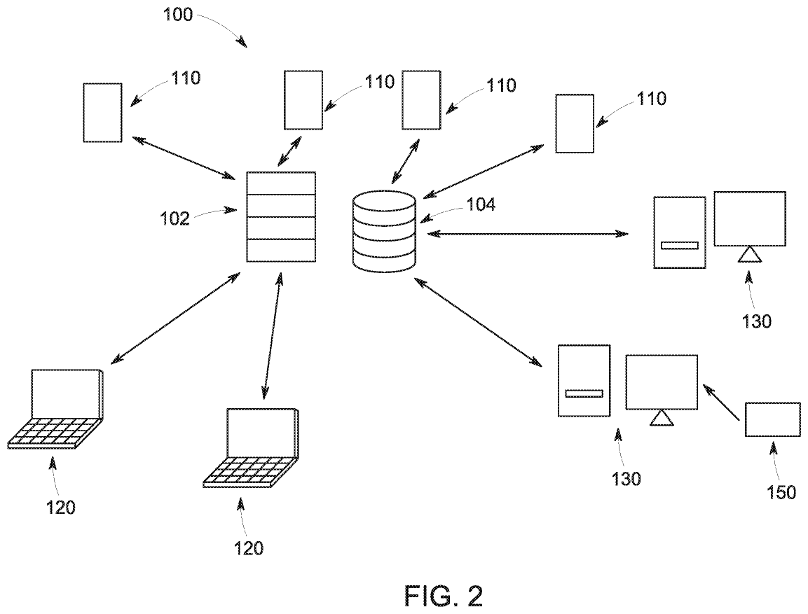

FIG. 3 shows one embodiment of a Service Provider User Interface 300 as it would appear on a service provider's user device (SPUD).

In this case the service provider that was chosen to illustrate the interface is Jane Peters, one of the physiotherapists discussed above.

The interface 300 presents Jane with her calendar 302, which is auto-filled with her appointments and which allows her to block out times that she is not available. As is common with calendars and electronic time management systems, the interface 300 allows Jane to select between daily, weekly, monthly mode etc. In this depiction it shows her calendar in scrollable daily mode.

Once a patient selects her for a session, e.g. at 8 am with James Robertson, and 6 pm with Emily Smith, on Monday, Sep. 7, 2020, this will appear on the calendar 302 together with the name and address of the patient. In this example, Jane has blocked out 10 am and 11 am on Monday, Sep. 7, 2020.

The interface 300 includes a start time 310 and end time 312 entry field for Jane to record when she commences the session and when she completes it.

In a notes section 320 of the interface 300, Jane can record the exercises done and include comments on the patient's, performance and/or improvements over the previous session.

In order to create a care plan, one implementation provides the patient's physician with a Medical Provider User Interface 400 on a Medical Provider User Device (MUD). The MUD may comprise a separate computing device (e.g. a smart phone with a mobile app defining the interface 400). In this embodiment, it is also integrated into the physician's electronic medical record (EMR) system which is supported on a server 150 (FIG. 1).

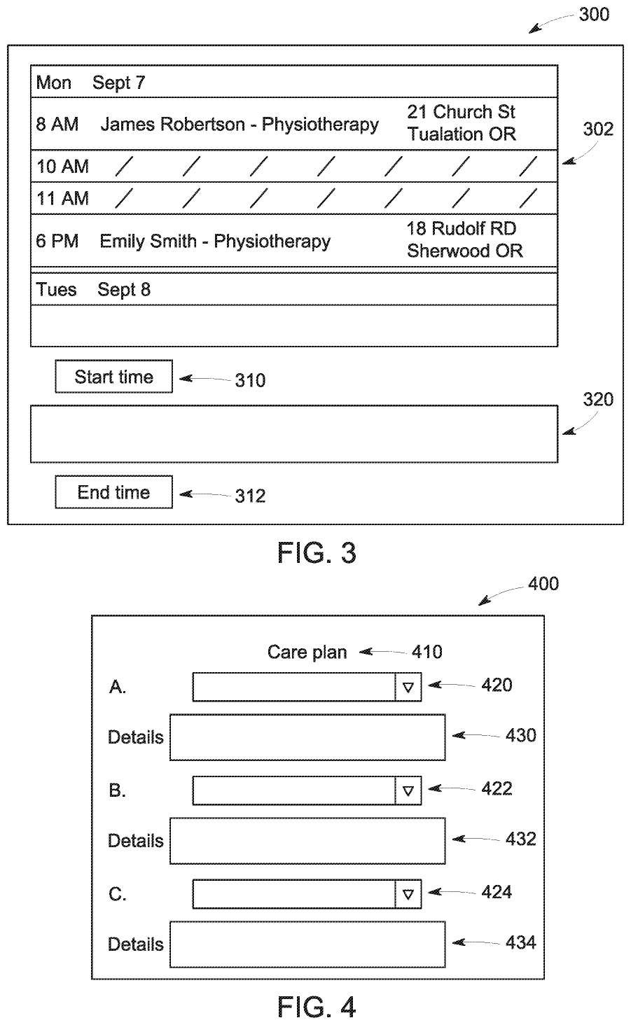

Referring again to the interface 400 of FIG. 4, the physician, as part of treating a patient, will record the details of the interaction with the patient. In this case this would include details about the hip surgery with Ms. Smith. The EMR in this case is supplemented with a care plan 410, which prompts the doctor by means of drop-down menus 420, 422, 424 to define the care plan, e.g. the need for physiotherapy (drop-down 420) (with a data entry block 430 to define details such as the frequency and the duration of the physiotherapy). In this example the doctor also proposes wound care (drop-down 422) with dressing changes every second day for the first 6 days, defined in a details block 432. The drop-down menu 424 provides prescriptions for all pharmaceutical products required by the patient, the data entry block 434 allows details to be entered, e.g., directions for use of the pain medication.

The MUD communicates with the server 102 to capture the care plan for Ms. Smith in the database 104. The information in the care plan 410 is parsed to define the service providers required: in this case a physiotherapist, a nurse, and delivery of a pharmacy prescription.

The parsing and processing of the data in one embodiment is done by a processor controlled by means of machine-readable code on a memory device connected to the processor, wherein the machine-readable code defines an algorithm to control the processor to communicate with the various user devices, save data to the database104, identify what service providers need to be notified and what services need to be performed.

In one embodiment this parsing of the data from the physician's EMR or MUD, the identifying of service providers, the presentation of possible service providers to the PUD for selection of specific service providers, and the notifying of service providers by sending a message to the selected SPUDs, is performed using an artificial intelligence (AI) system as discussed in further detail below.

In one embodiment, the service provider's user interface includes a registration page, which allows service providers to specify their name, address (to provide location information), the service they provide, their education level (upload of supporting documents), insurance companies they are contracted in with, and their time availability (which in this embodiment is defined by having each service provider fill in a calendar or providing a link to their calendar). The server 102 allocates all the service provider information to a service provider section of the database 104.

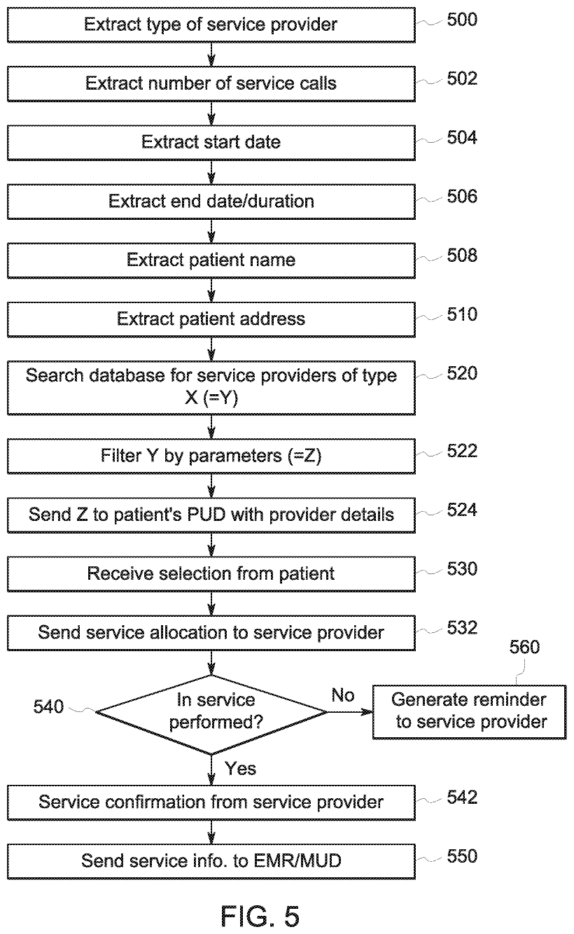

When a physician fills out care plan 410, the information is parsed by the processor (in this case server 102) by means of an algorithm stored in memory, which is in communication with the processor. As shown in FIG. 5, the algorithm identifies the various types of service providers involved (step 500), the frequency of service required (step 502), the start and end dates or duration of the service (steps 504, 506), the name of the patient (step 508), and the location of the patient (step 510). The algorithm then identifies all service providers in the database 104 corresponding to the type identified in step 500 (step 520), filters these by location of the patient (step 522) to identify specific service providers within a defined range or travel time of the patient, and presents these to the patient by sending a list of said service providers to the patient's PUD (the patient's device ID may be stored in the database 104 together with the patient information). The list of service providers is sent together with details about each service provider (step 524), which allows the patient to make a selection based on cost, availability, service location, and rating, as discussed above with respect to FIG. 2. As mentioned above, the algorithm may also take into account additional filters that the patient specifies, as part of the decision-making process before presenting the patient with a list of service provider options.

Once the user makes a selection, this is communicated back to the server 102 (step 530), which generates a message to the service provider's SPUD that automatically populates the service provider's user interface with the date, location, and service details, as discussed above with respect to FIG. 3 (step 532).

The processor then checks for confirmation on the specified start date, to confirm that the service was performed (step 540). Once the service provider performs the service and fills out the details (field 320 of FIG. 3), this is communicated back to the server 102 (step 542), which provides this information to the EMR or MUD (step 550) of the physician so that the physician can obtain confirmation that the service has been performed and maintain an up-to-date record about the patient in order to provide an overview of what elements in the care plan have been completed and to verify patient compliance with the care plan.

If, in step 540 the processor does not receive confirmation that the service was performed it sends a reminder to the service provider (step 560). If the service confirmation is still not received the processor may contact the patient's PUD with an apology message and request that the patient select an alternative service provider, which then repeats the steps from step 524.

In order to verify completion of a task by a service provide, the service provider may enter pertinent times and details of the services provided, and where possible, supplement this information with electronic sensor data, e.g., a digital image of the patients' wound prior to and after applying the new dressing. In another scenario, a service provider may be required to capture vital signs such as heart rate and blood oxygenation levels. These my be captured using electronic medical devices, and the results may be uploaded to the data memory via the service provider user interface on the SPUD.

While the present invention has been described with respect to specific embodiments and examples, it will be appreciated that the details of the algorithm, the types of data collected, the configuration of the system, and the layout and details of the various user interfaces may vary without departing from the scope of the invention as defined by the claims.

What is claimed is:

1. A system for implementing a care plan for a patient, comprising
a processor,
memory in communications with the processor and configured with machine-readable code to define an algorithm;
a data memory for storing data;
at least one patient user device (PUD) with patient user interface, in communication with the processor,
at least one service provider device (SPUD) with service provider user interface, in communication with the processor, and
at least one medical provider user device (MUD) with patient user interface, in communication with the processor.

2. The system of claim 1, wherein the data memory forms part of an information capture system, and the algorithm defines a decision-making system (also referred to herein as an Optimizer), to identify potential service providers based on parameters that include one or more of: availability, proximity to the patient, insurance coverage for the patient, customer rating profile, and gender of the service provider.

3. The system of claim 2, wherein Optimizer also takes into account the ability of a service provider to perform multiple required tasks forming part of a care plan.

4. The system of claim 2, wherein the Optimizer presents the patient on the patient user interface with several options of service providers based on one or more of: insurance coverage for the patient, availability, proximity, and rating, or identifies an optimal service provider based on a ranking of said parameters.

5. The system of claim 4, wherein the algorithm includes logic to verify compliance by the service provider in completing the one or more tasks allocated to the service provider.

6. The system of claim 5, wherein compliance by the service provider is assessed based on data uploaded by the service provider via the user interface on the service provider's SPUD, including one or more of: time and details of the task, and digital input captured by the service provider using one or more medical sensors.

7. The system of claim 4, wherein the Optimizer re-allocates a service provider in real time if a service provider selected by a patient becomes unavailable or fails to verify compliance.

8. The system of claim 4, wherein the algorithm includes logic to keep a record of tasks performed for a patient as part of the patient's care plan.

9. The system of claim 4, wherein the service provider user interface defines a scheduling and management system to schedule the next appointment and define what task(s) are to be performed and where they are to be performed.

10. The system of claim 4, wherein the patient user interface is arranged to provide the patient with an up-to-date overview of the tasks associated with a particular care plan and which tasks have been performed, as well as when, where, and through whom the next task(s) will be performed.

11. The system of claim 10, wherein the patient user interface is arranged to allow the patient to select and change service providers.

12. A system for implementing a care plan for a patient, comprising:
a physician portal configured to receive care plan information,
a service provider portal for entering service provider details and receive service requests,
a patient portal for selecting one or more service providers based on a care plan for the patient,
a processor,
a data memory, and
a control memory connected to the processor, wherein the control memory includes machine-readable code defining an algorithm for identifying suitable service providers for one or more tasks in the care plan and presenting them for selection to the patient via the patient portal.

13. The system of claim 12, wherein the portals are defined by a web application or native local app.

14. The system of claim 12, wherein the algorithm takes into account parameters that include one or more of: availability, proximity to the patient, insurance coverage for the patient, customer rating profile, and gender of the service providers.

15. A method of implementing a care plan for a patient, comprising
defining the tasks involved in the care plan,
identifying suitable service providers for each task based on one or more of: availability, proximity to the patient, insurance coverage for the patient, customer rating profile, and gender of the service provider,
allowing the patient to select a service provider for each task from the identified suitable service providers, and
tracking compliance by one or more of: the patient and the selected service providers.

16. The method of claim 15, wherein the defining of tasks in the care plan, the selection of service providers by the patient, and interaction with service providers is provided via an Internet server system that defines a user portal and a service provider portal.

17. The method of claim 16, wherein the Internet server system further defines a physician portal for capturing the tasks involved in the care plan.
